(19) United States
(12) Patent Application Publication
(10) Pub. No.: US 2010/0121274 A1
(43) Pub. Date: May 13, 2010

(54) PREFILLABLE CONSTANT PRESSURE AMBULATORY INFUSION PUMP

(75) Inventors: SEIK OH, Trabuco Canyon, CA (US); Kwan J. Kim, Fountain Valley, CA (US)

Correspondence Address: K&L Gates LLP, P.O. Box 1135, Chicago, IL, 60690-1135, US

(73) Assignees: BAXTER INTERNATIONAL INC., Deerfield, IL (US); BAXTER HEALTHCARE S.A., Zurich (CH)

(21) Appl. No.: 12/269,494

(22) Filed: Nov. 12, 2008

(51) Int. Cl. A61M 5/20

(52) U.S. Cl. 604/135

(57) ABSTRACT

A portable infusion device is provided that includes a cylindrical housing having a housing end, an inflatable cartridge sized to fit within the housing and a piston located within the housing. The cartridge can include a cartridge end having an inlet and an outlet. The cartridge end can be connected to an inflatable portion and be configured to be releasably secured to the housing end when the cartridge is inserted into the housing through the housing end. The piston can include at least one biasing device positioned to apply a constant force to the inflatable portion to expel a medical fluid from the cartridge.

### BACKGROUND

The present disclosure relates generally to portable infusion devices and more specifically to portable prefillable infusion devices. Infusion pumps are used commonly to deliver a wide variety of medication to medical patients. Infusion pumps are used to deliver, for example, intravenous fluids and solutions for medical therapies such as chemotherapy, antiviral and antibiotic therapy. Infusion pumps are used also to intravenously introduce blood, saline solutions, glucose solutions and other medical fluids including drugs and pharmaceuticals.

Besides a single infusion of medication to a patient, a patient may require multiple infusions on a daily basis, intermittent infusion over a time period, or even a slow, continuous introduction of medication into a patient. Specifically, certain medicinal therapies require the infusion of medication over a particular period of time that can range from a short period (about 30 minutes) to an extensive period (several days). It is important, therefore, to administer these medication doses completely and accurately. Accurate administration requires, for example, a consistent and controllable flow rate.

There is an increasing reliance on outpatient and home care treatment. Different infusion devices, however, have different drawbacks. Many existing infusion pumps do not offer the portability required to meet the needs of an ambulatory patient because these devices generally require a patient confined to a bed while others are too bulky to be an option for the ambulatory patient.

High-end infusion pumps contain sophisticated electrical components and mechanisms that are expensive. Other ambulatory infusion pumps use mechanical members that impart a dispensing force that is often inconsistent and inaccurate.

Still other infusion devices are single, self-contained units, such that the device, though refillable, must be disposed after the single medication therapy is complete. It is common practice to dispose fluid contacting components in medication infusion. In most single use infusion pumps, the container of the solution is integrated into the pump unit or the container itself serves as the energy source, especially in elastomer devices. Therefore, the fluid contacting components are inseparable from the pump unit, which consequently results in disposing the whole unit after the single medication therapy is complete.

A need accordingly exists for an ambulatory infusion pump device that operates simply and inexpensively. A need also exists for an ambulatory infusion pump device that dispenses a uniform flow rate of medication and that can dispense multiple types of medication without disposal of the entire device.

### SUMMARY

The infusion devices of the present disclosure provide portable, reusable, non-electrical, infusion devices that can consistently dispense medications with a uniform flow rate multiple times to a patient. In one embodiment, for example, the device includes an inflatable cartridge that secures a bellows inside of a housing. The housing has a piston that applies a constant force to the bellows to dispense medical fluid to a patient at a uniform flow rate.

The infusion device can include a cylindrical housing and an inflatable cartridge sized to fit within the housing. The cartridge includes an inlet and an outlet and an end configured to be releasably secured to an end of the housing when the cartridge is inserted into the housing. The cartridge end is also connected to an inflatable portion of the cartridge. The infusion device also includes a piston that is located within the housing. The piston includes at least one biasing device or spring positioned to apply a constant force to the inflatable portion of the cartridge to expel a medical fluid from the cartridge.

In a further embodiment the cartridge may be placed in a barrier over-wrap containing a desired gas to increase the shelf life or performance of the infusion device.

In another embodiment of the present disclosure, a portable infusion device is provided.

In still another embodiment of the present disclosure, a portable infusion kit is provided. The infusion kit includes a portable infusion device and a retracting tool. The infusion device can have a cylindrical housing and an inflatable cartridge filled with medical fluid. The inflatable cartridge includes an inflatable portion, which is sized to fit within the housing, while an end of the cartridge is releasably secured to the housing when a bellows is inserted into the housing. The infusion device further includes a piston located within the housing. The piston includes at least one biasing device or spring positioned to apply a constant force to the inflatable portion of the cartridge to expel the medical fluid from the cartridge.

The retracting tool includes a threaded rod, which includes a handle on one end and a nut configured to be releasably secured to the housing.

It is, accordingly, an advantage of the present disclosure to provide a portable infusion device that is refillable with the same medication for the same patient.

It is another advantage of the present disclosure to provide a portable infusion device that administers medication at a uniform flow rate.

It is a further advantage of the present disclosure to provide a portable infusion device that can be reused to administer different medications.

It is yet another advantage of the present disclosure to provide a portable infusion kit for administering different medications within a single housing.

It is yet a further advantage of the present disclosure to provide a portable infusion kit with prefillable cartridge having a reloading kit.

It is still a further advantage of the present disclosure to provide a ambulatory infusion device that is significantly smaller and lighter than existing reusable ambulatory infusion pumps.

It is another advantage of the present disclosure to provide adequate compatibility with most of the drugs currently administered with ambulatory infusion pumps.

It is a further advantage of the present disclosure to provide an a prefilled ambulatory infusion pump which maintains an extended shelf life.

It is a further advantage of the present disclosure to provide an accurate means of estimating the remaining volume or the dispensed volume of the medication during infusion.

It is yet another advantage of the present disclosure to provide a flow indicating device, especially for viewing very slow flow rate infusion.

It is yet a further advantage of the present disclosure to allow the device to be prefilled.

Additional features and advantages are described herein, and will be apparent from the following Detailed Description and the figures.

### BRIEF DESCRIPTION OF THE FIGURES

FIG. 1 is a cross-section view of a disassembled embodiment of an infusion device of the present disclosure.

FIG. 2 is a cross-section view of the cartridge embodiment of FIG. 1 with a compressed bellows.

FIG. 3 is a perspective view of the cartridge cap embodiment of FIG. 2.

FIG. 4 is a perspective view of the piston embodiment of FIG. 1 that contains two negator springs.

FIGS. 5A to 5C are partial cross-section views illustrating the connection of the pump and the cartridge and expansion of the bellows according to one embodiment of the present disclosure, in which the pump is in cross-section and the cartridge is in full view.

FIG. 6 is a perspective view of one embodiment of a portable infusion device of the present disclosure.

FIG. 7 is a perspective view of the portable infusion device embodiment of FIG. 6 with multiple cartridges.

FIG. 8A is a perspective view of one embodiment of an infusion device with a flow indicator of the present disclosure. FIGS. 8B and 8C are enlarged front views of the flow indicator of FIG. 8A.

FIG. 9 is a top view of the pump embodiment of FIG. 1.

### DETAILED DESCRIPTION

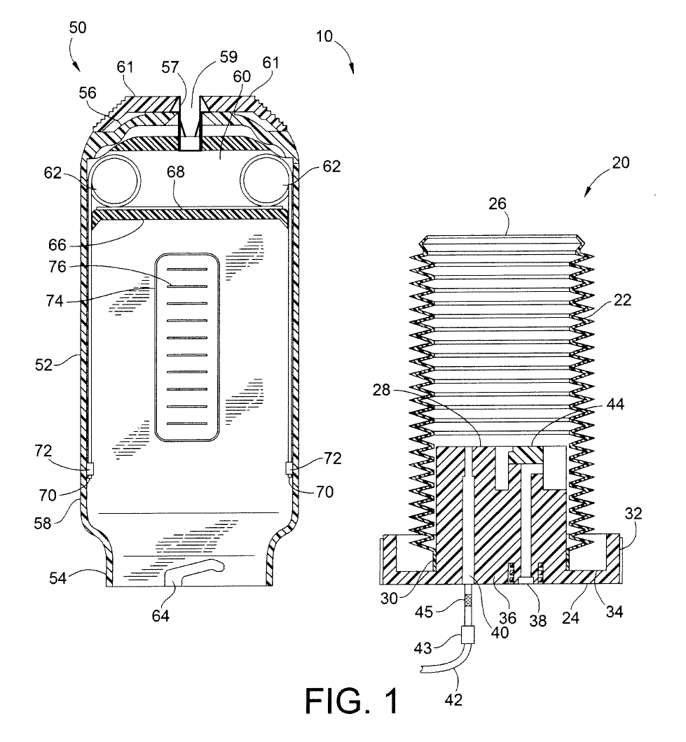

Referring now to the drawings, FIG. 1 illustrates one embodiment of a portable infusion device 10. Infusion device 10 includes a compressible cartridge 20 and a reusable pump 50. As will be described in more detail below, cartridge 20 is configured to fit inside pump 50 such that pump 50 can dispense medical fluid contained in cartridge 20.

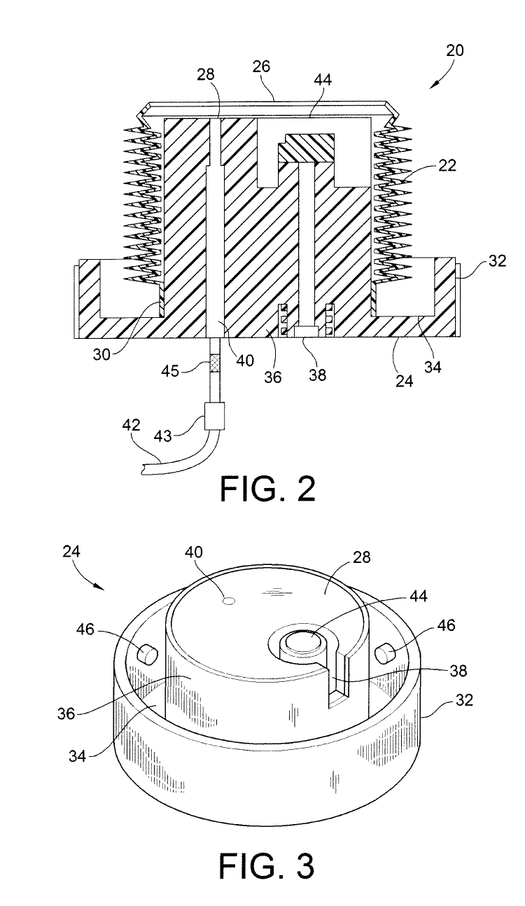

Compressible cartridge 20 is generally cylindrical, disposable and includes a compressible bellows 22 and an end or cap 24. Bellows 22 includes a top surface 26. Bellows 22 has an accordion-like structure as shown in an expanded configuration in FIG. 1, in which a hollow interior of bellows 22 is filled with a drug or pharmaceutical composition. When not filled with a medication or after expelling medication, bellows 22 assumes a compressed configuration as shown in FIG. 2.

Bellows 22, in one embodiment, is made of low-density polyethylene (“LDPE”), which is known to be chemically inert and compatible with most drugs. The material used to make bellows 22 can also be multi-layered to provide increased moisture barrier properties and/or for mechanical strength enhancement to cartridge 20 when pre-filled with medication.

The angle of each corrugation of expanded bellows 22 is about 60° in the illustrated embodiment. Narrowing the corrugation angle provides bellows 22 with more corrugations, which therefore provides bellows 22 with more resistance against bursting or buckling when expanded. If the corrugation angle is too narrow, however, then bellows 22 requires significantly more corrugations to achieve the same volume as that shown in the illustrated embodiment. Consequently, the height of bellows 22 increases when fully compressed, which increases the potential dead volume, and the length of the device.

The diameter of bellows 22 is sized as needed to provide sufficient stability to maintain rigidity and straightness of bellows 22 and to prevent buckling or bending of expanded bellows 22 when under pressure. Increasing the diameter improves buckling resistance, but necessitates a greater force to compress bellows 22 to expel fluid at a required flow rate. The wall thickness of bellows 22 is provided to prevent bursting or flattening of the corrugation under maximum positive pressure. If the wall thickness is too thick, the height of the compressed bellows will increase, which increases the potential dead volume within the compressed bellows.

Bellows 22 may also be made of other flexible materials such as polyolefin or an elastomer or rubber. If the bellows is made of a material such as an elastomer capable of being inflated or stretched to the filled configuration, use of the corrugations may not be needed.

Cap 24 connects cartridge 20 to pump 50 and closes bellows 22 to hold medication contained in the interior of bellows 22. Cap 24, in one embodiment, is injection molded from high-density polyethylene (“HDPE”) to provide welding compatibility with the mating bellows (made of “LDPE”) while providing structural strength. Cap 24 is made alternatively from a polyolefin, such as polypropylene.

Cap 24 includes three main portions: an outer rim 32, a bottom surface 34 and a center boss 36. A flange 30 on bellows 22 is affixed to cap 24 at center boss 36 as illustrated in FIG. 1. Outer rim 32 provides a handle on cartridge 20 for assembling (connecting) and disassembling (disconnecting) cartridge 20 to and from reusable pump 50. The exterior surface of outer rim 32 is corrugated to prevent slippage when rotating cartridge 20. The interior surface of outer rim 32 includes, for example, three or four cylinder shaped lugs 46 (shown in FIG. 3) extending in and from the interior surface. Lugs 46 have the size and shape necessary to engage mating locking grooves 64 on pump 50 when connecting cartridge 20 to pump 50.

Center boss 36 of cap 24 includes an inlet 38 having a check valve 44 (best seen on FIG. 3), an outlet 40 and an associated tube 42 with a flow restrictor 43 and an inline air eliminating filter 45. Flow restrictor 43 is configured to restrict flow out of outlet 40 to a desired flow rate over the course of an infusion. When opened, restrictor 43 has a small diameter opening permitting fluid flow between outlet 40 and the outlet of tubing 42. Illustrated inlet 38 is a female luer port that can accept a syringe or male luer for the injection of medication. Illustrated outlet 40 is a flow channel communicating with tube 42. Filter 45, located in tube 42 upstream from flow restrictor 43, eliminates the potential for air bubbles and the generation and migration of particulate matter during the filling procedure.

Center boss 36 extends from the bottom surface 34 of cap 24 to the height of a fully compressed bellows 22 (shown in FIG. 2) such that bellows 22, when fully compressed, contacts center boss 36 at a cap head 28. Center boss 36 fills the dead volume of fully compressed bellows 22 to minimize the residual volume of, for example, an expensive drug at the completion of infusion when bellows 22 is at maximum compression. The diameter of center boss 36 is slightly smaller than the inner diameter of compressed bellows 22, so that bellows 22 can be compressed and expanded freely without any interference from center boss 36.

Cartridge 20, including bellows 22, cap 24 and outlet tube 42 are all part of the fluid contacting portion of infusion device 10. These portions generally are not refilled after use and, instead, are discarded after use. However, for the same patient using the same medication, cartridge 20 can be refilled multiple times when a large volume therapy is preferred. For example, a cartridge of 60 mL size can be refilled five times for 300 mL total therapy when only 60 mL size device is available, thereby providing a pump that consistently dispenses fluid during repeated use.

Reusable pump 50 as illustrated in FIG. 1 includes a generally cylindrical housing 52 having an open end 54, a top end 56 and a piston 60 located within housing 52, which is operated by two constant force springs 62 (or negator springs). Housing 52 can be injection molded from a rigid plastic like polycarbonate, polyester or acrylonitrile butadiene styrene (“ABS”), which provide structural integrity to withstand a high-tension force exerted by negator springs 62, while being sufficiently clear to see inside the device through a provided window.

The cylindrical shape of housing 52 extends from open end 54 to top end 56, and has an inner diameter that is slightly bigger than the outer diameter of bellows 22, so that piston 60 (with springs 62) and bellows 22 can slide along the inside wall of housing 52 with minimal resistance. The clearance space between the inside wall of housing 52 and piston 60/bellows 22 is minimized, however, to prevent buckling of bellows 22 or wiggling of piston 60 as each moves up and down along housing 52.

Housing 52 also includes a spring path 58 having a rectangular shape extending outward from the main cylindrical portion of housing 52. Spring path 58 provides a path for the coiling and uncoiling of negator springs 62 as discussed in detail below. The height and the width of the rectangular shape spring path 58 are determined by the thickness and the width of the strap of the negator springs. Open end 54 also includes mating grooves 64 located on the edge of open end 54. Mating grooves 64 are configured to mate with inserted cartridge 20 as described herein.

Housing 52 can be opaque or translucent, for example, via colored plastic or textured surface treatment. Two viewing windows 74 may be installed on both walls of housing 52 so that the fluid content within bellows 22 and the movement of bellows 22 can be seen from the outside of housing 52. One of the illustrated viewing windows 74 includes fine graduation marks 76 printed to estimate the residual medicament volume during infusion of medication by compression of the bellows. By providing an indicating line (not shown) on the side wall of piston 60, the pump's residual volume can be estimated accurately to the milliliter by noting the indicating line's location relative to the graduation marks during the linear movement of bellows 22 within pump 50.

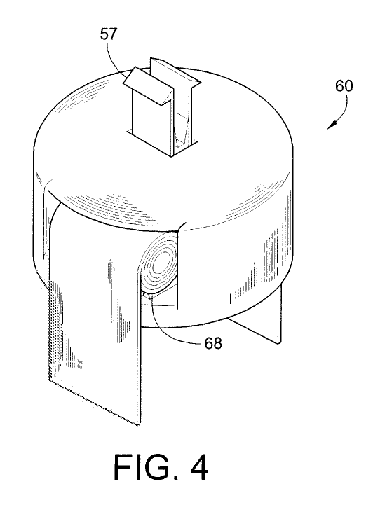

Referring again to FIG. 1, piston 60 includes a bottom surface 66 and two parallel ribs 68 (best seen on FIG. 4) extending upward from bottom surface 66 of piston 60. The railroad-shaped ribs minimize piston surface contact with the surface of the negator springs, which consequently allows a free movement of negator springs 62 as they coil and uncoil. The size and shape of bottom surface 66 of piston 60 is such that top surface 26 of bellows 22 can snap-fit to bottom surface 66. For example, the flat portion of bottom surface 66 may have a width and length that is slightly larger than the length and width of top surface 26 of bellows 22 such that the top surface 26 snap-fits into bottom surface 66 of piston 60. The diameter of piston 60 is slightly larger than the diameter of compressed bellows 22 so bellows 22 can be compressed and expanded with no interference with springs 62. Piston 60, including bottom surface 66 and ribs 68, can be injection molded from a plastic of low friction coefficient like Polyacetal, or any thermoplastic material that can provide rigid structural integrity. The piston can also include a lubricant or coating for minimizing friction.

The two negator springs 62 made, for example, of stainless steel, lie within piston 60 on two parallel protruding ribs 68 (better shown in FIG. 4). Negator springs 62, when uncoiled, exert a constant compression force on bellows 22 regardless of the displacement of the springs. Ribs 68, protruding upwardly from surface 66 of piston 60, allow springs 62 to rotate or slide freely within piston 60 with minimal frictional resistance. Each spring 62 also includes a spring tip 70 that is fixed to housing 52 at an attachment portion 72. Attachment portions 72 reside on the inside surface of housing 52, specifically in spring path 58 of housing 52. Moreover, in one embodiment, low friction material such as Teflon™ lubricates or coats the outside surface of negator spring 62 to promote unimpeded coiling and uncoiling of springs 62 and unimpeded movement of springs 62 within piston 60 or, at minimum, movement and coiling/uncoiling with minimal frictional resistance. Other low friction materials include, for example, PTFE coating (FluroMed®) or vapor deposition Parylene.

The dimension (strength) of spring 62 is determined by the required pressure of the solution retained within bellows 22 of cartridge 20. The required pressure is based on the desired flow rate and viscosity of the medication, and the pressure differential between the upstream pressure of flow restrictor 43 and the downstream physiological backpressure provided by the medication within bellows 22. The tension (or retracting) force of each negator spring 62 is determined by multiplying the required pressure by the cross-sectional area of bellows 22, and dividing that value by the number of springs, which is two in the embodiment illustrated in FIG. 1. The width and diameter of the coiled spring are optimally determined from a table of values from the spring supplier, or can be custom designed if necessary. Currently, most of the disposable ambulatory infusion pumps are designed to exert the pressure from about 4 psig to about 9 psig. With these fixed pressures, flow rate is adjusted by the dimension (bore diameter and the length) of the outlet or tubing instead of varying the pressure of the pump. To overcome a high backpressure, the present disclosure advantageously provides a higher pressure using a stronger spring.

Reusable pump 50, including housing 52, springs 62 and piston 60, are part of the non-fluid contacting portion of infusion device 10. Therefore, these non-fluid contacting portions are reusable, and do not need to be disposed after use for contamination reasons.

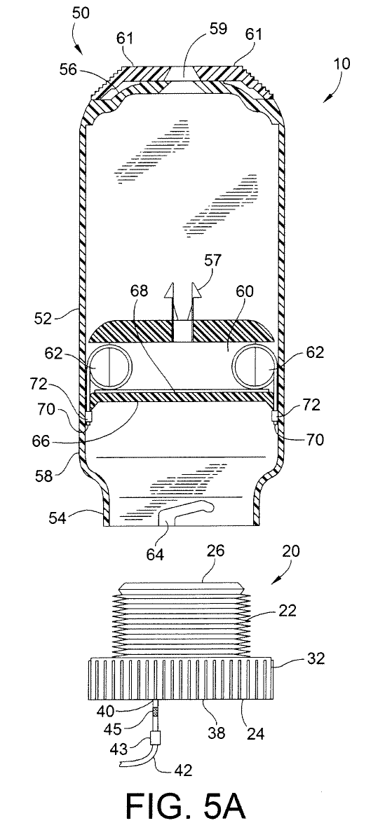

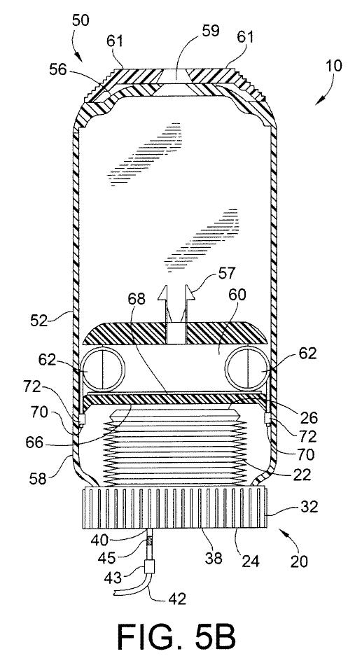

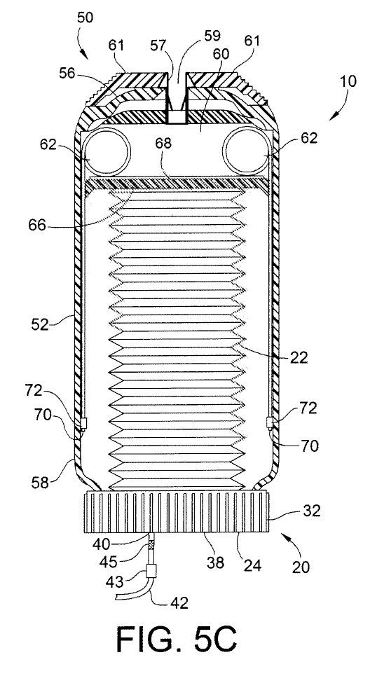

Springs 62, and corresponding piston 60, reciprocate between a resting, coiled position adjacent attachment portion 72 of housing 52, and a non-resting, uncoiled position near top end 56 of housing 52. FIG. 1 illustrates springs 62 in a non-resting, uncoiled position, in which springs 62 have uncoiled, due to positive fluid pressure within bellows 22, via spring tips 70 held to housing 52. FIG. 5A, on the other hand, shows springs 62, and piston 60, in a resting, coiled position.

In the resting, coiled position of FIG. 5A, piston 60 and springs 62 rest inside housing 52 adjacent attachment portion 72 of the housing. In this resting position, the springs are not stretched and therefore have no stored kinetic energy. In FIG. 5A, cartridge 20 is shown outside of and not connected to pump 50. Bellows 22 of cartridge 20 is fully compressed because no medication is contained within the bellows.

To place infusion device 10 in a position to dispense medication to a patient, cartridge 20, with compressed bellows 22, is first engaged to pump 50 as illustrated in FIG. 5B. To engage cartridge 20 with pump 50, bellows 22 is inserted into pump 50 through open end 54 of pump 50. Bellows 22 is inserted into pump 50 until open end 54 of pump 50 contacts bottom surface 34 of cap 24 on cartridge 20. After contact, the user rotates cap 24 until lugs 46 (shown in FIG. 3) on cap 24 align with the entrance of mating grooves 64 (shown in FIG. 5A) on housing 52. Further rotation and slight pushing of cap 24 locks lugs 46 into place in mating grooves 64, thereby locking cartridge 20 to pump 50. When lugs 46 pass the peak of the mating groove (illustrated in FIG. 5A), the lug is snapped in a secure lock position. Slight tension is generated in this position, which enhances secure ‘lock’ without possibility of loosening of the cap. In this locked position, compressed bellows 22 and center boss 36 on cap 24 reside within housing 52 of pump 50. Top surface 26 of bellows 22 may contact bottom surface 66 of piston 60 or snap-fit to bottom surface 66 depending on (a) the location of piston 60 relative to bellows 22 and, as discussed previously, (b) the size and shape of piston bottom surface 66 relative to bellows 22. Other than the slight tension described above, bellows 22 does not impart any force on piston 60, nor does bellows 22 push piston 60 from its resting, coiled position illustrated in FIGS. 5A and 5B.

To inflate bellows 22 as shown in FIG. 5C, a user inputs medication into bellows 22 through inlet 38 using, for example, a syringe or filling machine (not shown). Fluid dispensed from the syringe or filling machine through the inlet applies sufficient pressure to open check valve 44 and allow the fluid to pass check valve 44 into bellows 22.

As medication is dispensed into collapsed bellows 22, bellows 22 inflates and applies pressure to piston 60. As bellows 22 inflates, springs 62 uncoil along spring paths 58 of housing 52. As springs 62 uncoil, piston 60 moves upward through housing 52 while spring tips 70 stay fixed to housing 52 at attachment portions 72. As springs 62 uncoil, piston 60 applies downward pressure to bellows 22. However, no fluid will dispense through outlet 40 as long as an end clamp or slide clamp (not shown) on tube 42 is activated. Moreover, as long as the infusion of fluid into bellows 22 continues, piston 60 will continue to move upward and springs 62 will continue to uncoil until piston 60 reaches top end 56 of housing 52 or filling of specified volume is completed, which can be smaller than the volume of the infusion device.

Once filling of medication is stopped, check valve 44 closes to prevent a backflow of fluid from escaping cartridge 20 through inlet 38. The infusion device is now ready to dispense drug through outlet 40. To commence dispensing, the end cap or slide clamp (not shown) is removed from tube 42, thereby allowing medication to flow. Springs 62 impart a constant recoiling force on bellows 22, dispensing medication at a constant flow rate. The flow rate should remain constant regardless of how far bellows 22 displaces piston 60 from its resting position illustrated in FIG. 5A. To allow for this constant force by springs 62, the springs rest on ribs 68, and uncoil and recoil along spring path 58, which allow springs 62 to rotate and slide freely with minimal frictional resistance.

Alternatively, a locking mechanism located at top end 56 of housing 52 may control dispensing of the drug. In this case, once infusion of medication into bellows 22 forces the piston to top end 56 of housing 52, a built-in latch 57 on piston 60 has two outward-protruding fingers that snap into a mating slot 59 at top end 56 of the housing and lock piston 60 to housing 52 with springs 62 fully uncoiled. Locked piston 60 prevents application of force on expanded bellows 22 even though springs 62 are uncoiled. To dispense the medication contained in bellows 22, a releasing mechanism disconnects built-in piston latch 57 from housing 52 to allow springs 62 to compress bellows 22 and dispense the medication. The releasing mechanism can include, for example, two sliding tabs 61 formed on the outside of housing 52 that is easily accessible by the user as illustrated in FIG. 1 and configured to slide toward each other to press the fingers of latch 57 towards each other to unlock and activate piston 60.

After dispensing the medication from bellows 22, the user discards cartridge 20 by rotating cap 24 in a direction opposite the initial engagement rotation direction to release lugs 46 of cartridge 20 from mating grooves 64 on housing 52. This motion releases cartridge 20 from pump 50 and allows pump 50 to accept a new cartridge 20.

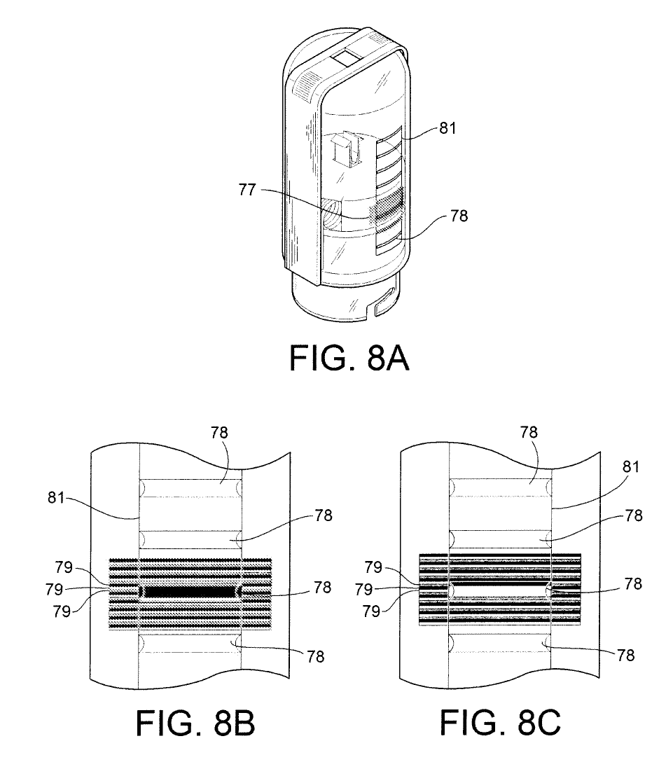

In the cases where fluid flow out of infusion device 10 is slow (e.g., 0.5 mL/hr), it can be difficult for a patient or a caretaker to check whether the solution is flowing or stopped by blockage. In those cases, portable infusion device 10 may also include a flow indicator mechanism as illustrated in FIG. 8A. The flow indicator includes a sticker label 77 attached to the side wall of piston 60 and a plurality of half cylinder shaped magnifying lenses 78 formed on a window 81 of housing 52. Sticker label 77 can include, for example, a color spectrum. A plurality of color bars 79 on sticker label 77 represent the color spectrum, with the width of each color bar 79 being as small as about 0.010″. The width of the sticker label is approximately 0.5″ and the length of the sticker is slightly bigger than the width of window 81.

The plurality of magnifying lenses 78 are installed across the length of window 81, which is installed on the opposite side of viewing window 74. Each magnifying lens 78 can have dimensions measuring, for example, 0.080″ in thickness and 0.75″ in length. Distance between each magnifying lens 78 is approximately 0.5″. The distance from the bottom surface of the lens to the sticker determines curvature of the magnifying lens, so the color spectrum of 0.010″ is magnified to fill entire width of lens, 0.080″. Magnifying lenses 78 are injection molded and have a clear appearance while the surrounding body of housing 52 are textured or colored opaque.

As piston 60 moves slowly downward along housing 52, sticker label 77 and corresponding color bars 79 pass underneath magnifying lenses 78. Magnifying lenses 78 magnify color bars 79 on sticker label 77, indicating clearly which of the color bars 79 lie beneath a respective magnifying lens 78, as illustrated in FIG. 8B. As piston 60 advances further down housing 52, sticker label 77 also advances along the same magnifying lens 78, indicating clearly a different color bar 82 or combination of color bars 79 lying beneath the same lens 78, as illustrated in FIG. 8C. For a flow rate of 5 ml/hr, for example, the color changes every 3 minutes to reflect the movement of piston 60. Further, for a flow rate of 0.5 ml/hr, the color changes about every 30 minutes. Therefore, these color changes provide a relatively inexpensive visual indicator that flow is occurring.

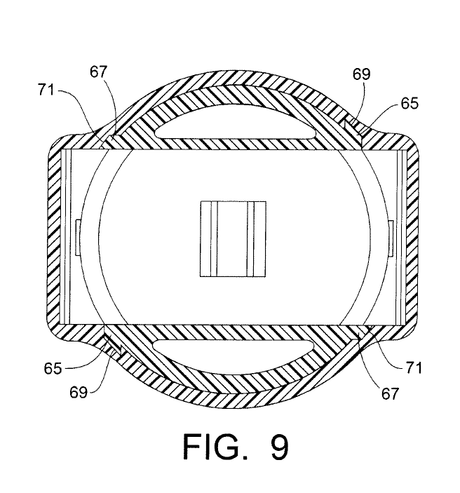

In another embodiment illustrated in FIG. 9, the side of piston 60 includes a pair of primary rails 65 and a pair of secondary rails 67. Each primary rail 65 is sized to fit into a primary groove 69 on housing 52 such that piston 60 can slide vertically through housing 52 without tilting off of vertical. Each secondary rail 67 is sized to fit into a secondary groove 71 on housing 52 such that piston 60 cannot rotate or slide horizontally within housing 52. By providing two sets of rails and corresponding grooves, piston 60 can slide smoothly and vertically within housing 52 and negator springs 62 can coil and uncoil within spring path 58 without any non-vertical movement.

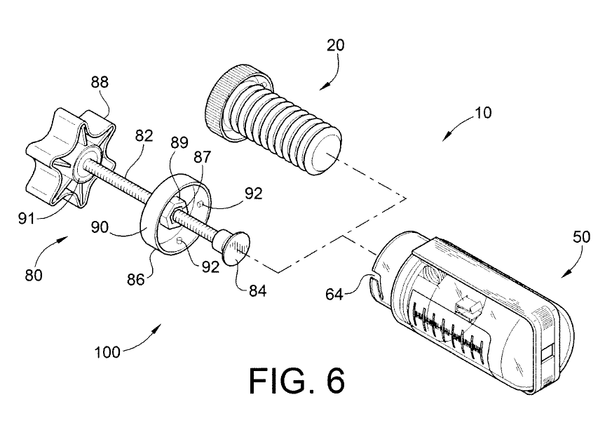

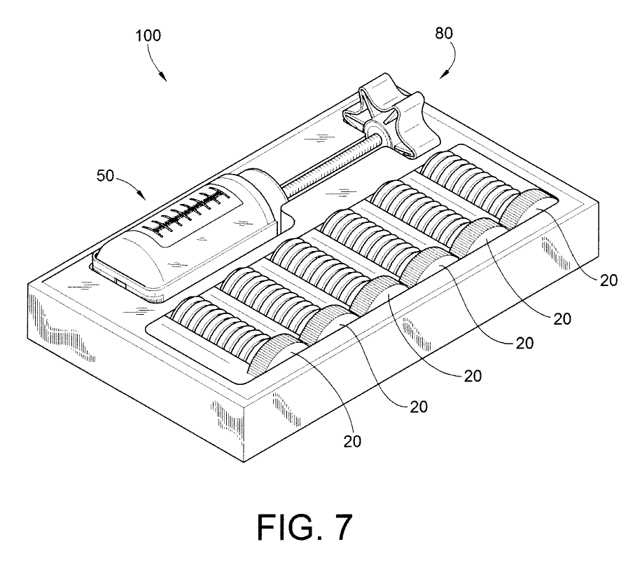

In an alternative embodiment illustrated in FIG. 6, a portable infusion kit 100 is provided. Kit 100 includes infusion device 10 and a retractor 80. Infusion device 10, as described above, includes compressible cartridge 20 and reusable pump 50. Alternatively, kit 10 may include a plurality of compressible cartridges 20 as illustrated in FIG. 7. Since cartridge 20 is fluid contacting and is therefore discarded after a single use, kit 10 provides a plurality of compressible cartridges 20 to allow for multiple uses of reusable pump 50 over multiple treatments.

Referring to FIG. 6 in an alternate embodiment, the cartridge 20 may be filled with a medication or other fluid and stored for a period of time before use. It has been found that the performance of the infusion device 10 may be affected by long-term storage. Some possible reasons are the transmission of water vapor through the wall of the bellows 22 or outlet tube 42 (FIG. 1) that may lead to some deterioration of the material of the bellows or may increase the concentration of the drug in the tube to where the drug will fall out of solution and crystallize to form a blockage. Other performance factors include the potential that oxygen or other atmospheric gas may migrate through the bellows 22 and have a detrimental effect on the fluid contained in the cartridge 20. A still further performance factor may be the affect that an atmospheric gas such as oxygen may have on the material of the cartridge 20. If the material of the bellows 22 is in a stressed state, such as when the material is an inflated elastomer, the effect of oxidation may be pronounced.

To protect the cartridge 20 and/or the contents and provide increased shelf life the cartridge may be packaged in an overwrap barrier container (not shown) to encase the cartridge in an enclosure which forms a gas barrier. Such a container may comprise aluminum film or foil or a polymeric film such as the outer envelope film described in U.S. Pat. No. 6,007,529, the disclosure of which is incorporated herein.

The atmosphere within the barrier container may be an atmosphere or an inert gas such as nitrogen or other gas that is more compatible with the contents of the cartridge or the material of the bellows 22 or a mixture thereof. In addition the moisture level of the atmosphere within the container may be selected to increase the performance characteristics of the infusion device 10. In an alternate embodiment the entire infusion device 10 may be stored within the overwrap barrier container.

In FIG. 6, retractor 80 includes a threaded rod 82, a contact surface 84, a mating nut 86 and a handle 88. Mating nut 86 includes a hex nut 87, hex nut housing 89 sized to fit hex nut 87, rim 90 and plurality of nut lugs 92 provided on the inside surface of rim 90. Nut lugs 92 have the same features and design as lugs 46 on cap 24, illustrated in FIG. 3, such that nut lugs 92, like lugs 46, can be locked to mating grooves 64 on housing 52 of pump 50. Handle 88 includes a rod fitment 91 with a hole sized to fit and hold threaded rod 82. Retractor 80, like pump 50, is non-fluid contacting and reusable. Rod 82 and mating nut 86 can be made from anodized aluminum, and can be made from a polyacetal material or any thermoplastic materials that provide sufficient mechanical strength with low friction coefficient. Handle 88 and contact surface 84 is made of acrylonitrile butadiene styrene (“ABS”).

If cartridge 20 is pre-filled with medication, springs 62 on pump 50 need to be retracted and locked at top end 56 of housing 52 so that cartridge 20 can be loaded into pump 50 without having to retract piston 60 using the already expanded bellows 22 of cartridge 20. To retract springs 62, retractor 80 engages pump 50, and forces springs 62 open (retracts the springs) to lock piston 60 in the forced-open or retracted position before inserting pre-filled cartridge 20.

Retractor 80 is fixed to housing 52 in much the same way as cap 24 on cartridge 20, illustrated in FIG. 5B. Specifically, mating nut 86 has substantially the same configuration as cap 24 such that, with threaded rod 82 in a fully retracted position, nut lugs 92 lock to mating grooves 64 on housing 52 as mating nut 86 is rotated onto housing 52.

Once retractor 80 is fixed to pump housing 52, the operator turns handle 88 to extend retracted threaded rod 82 into housing 52 such that contact surface 84 engages and pushes piston 60 up towards top end 56 of housing 52 while mating nut 86 remains fixed to mating grooves 64 on housing 52. At top end 56, built-in latch 57 on piston 60 snaps into a mating slot 59 located at top end 56 to lock piston 60 to housing 52 with springs 62 fully uncoiled. The operator then turns mating nut 86 of retractor 80 in the opposite direction to disengage the retractor from pump 50 by unlocking from mating grooves 64. The operator then fits pre-filled cartridge 20 into pump 50 and locks it into position by engaging lugs 46 on cartridge cap 24 with mating grooves 64 on housing 52. The threaded rotation to translational motion of retractor 80 provides even a weak patient with the requisite mechanical power to retract piston 60 and load cartridge 20.

Alternatively, retractor 80 can retract piston 60 by pressing pump 50 onto retractor 80, which is fixed to a flat surface, causing contact surface 84 to face upward. Using body weight, a user presses pump 50 down on contact surface 84 of retractor 80 to allow contact surface 84 to contact bottom surface 66 of the piston to translate piston 60 and uncoil springs 62 to top end 56 of housing 52, locking piston 60 to housing 52 as described above. Here, a user with sufficient strength does not need to lock retractor 80 to pump 50 and rotate handle 88 to move the piston.

To dispense the medication contained in bellows 22, releasing mechanism 61 previously discussed disconnects the built-in latch 57 from housing 52 to allow springs 62 to compress bellows 22 and dispense the medication. After dispensing the medication from bellows 22, the user discards cartridge 20 as discussed above because it is the fluid-contacting part of infusion device 10, or the user can refill the cartridge with the same medication for longer and larger infusions.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A portable infusion device comprising:
a cylindrical housing including a housing end;
an inflatable cartridge sized to fit within the housing, the cartridge including a cartridge end having an inlet and an outlet, the cartridge end configured to be releasably secured to the housing end when the cartridge is inserted into the housing through the housing end, the cartridge end connected to an inflatable portion of the cartridge;
a piston located within the housing, the piston including at least one biasing device positioned to apply a constant force to the inflatable portion of the cartridge to expel a medical fluid from the cartridge.

2. The portable infusion device of claim 1, wherein the at least one biasing device includes a spring that is normally coiled and that is uncoiled to be able to apply the constant force to the inflatable portion to expel the medical fluid from the cartridge.

3. The portable infusion device of claim 1, the inflatable portion of the cartridge and the piston configured such that filling the inflatable portion with the medical fluid moves the piston to a retracted position so that the piston can thereafter apply the constant force to the inflatable portion to expel the medical fluid from the cartridge.

4. The portable infusion device of claim 3, the housing including a locking mechanism that maintains the piston in the retracted position.

5. The portable infusion device of claim 1, which includes a tube provided at the outlet of the cartridge to form a path for the expelled medical fluid.

6. The portable infusion device of claim 5, the tube including a flow restrictor provided on the tube to maintain a desired flow rate over the course of infusion.

7. The portable infusion device of claim 1, the inlet including a check valve provided to close the inlet and prevent reverse of flow.

8. The portable infusion device of claim 1, the piston configured to be pushed by an external device to bias the at least one biasing device and place the piston in a retracted position prior to insertion of the cartridge into the housing.

9. The portable infusion device of claim 1, the inflatable portion including a bellows.

10. The portable infusion device of claim 1, the housing including a window that is substantially transparent such that the medical fluid is viewable from outside the housing.

11. The portable infusion device of claim 1, the housing including a volume indicator that accurately estimates infused or remaining drug with 1 mL resolution.

12. The portable infusion device of claim 1, the housing including a fine color spectrum magnified with a plurality of lenses.

13. The portable infusion device of claim 1, wherein the cartridge end includes a cap sealed to the inflatable portion of the cartridge.

14. The portable infusion device of claim 1, the housing including mating grooves formed on the housing end, the cartridge end including lugs, wherein the mating grooves and lugs are sized and shaped to releasably secure the cartridge end to the housing end when the cartridge is inserted into the housing through the housing end.

15. The portable infusion device of claim 1, the piston including two constant-force springs positioned to apply the constant force to the inflatable portion of the cartridge to expel the medical fluid from the cartridge.

16. The portable infusion device of claim 15, the housing including spring paths sized and shaped to allow the springs to ravel and unravel with minimal interference from the housing.

17. The portable infusion device of claim 16, wherein the spring paths are rectangular-shaped.

18. The portable infusion device of claim 15, the piston including a pair of rails sized and shaped to engage with rail grooves installed on a side wall of the housing to allow the vertical movement of the constant force springs with minimal resistance.

19. The portable infusion device of claim 15, wherein the constant force springs are placed inside the piston so that the springs can slide freely within the piston.

20. The portable infusion device of claim 15, wherein constant force springs are coated with low friction material to promote unimpeded movement of the springs within the piston.

21. A portable infusion device comprising:
a cylindrical housing;
an inflatable cartridge including a bellows connected to an end of the cartridge, the bellows sized to fit within the housing, the end of the cartridge configured to be releasably secured to the housing when the bellows is inserted into the housing, and
a piston located within the housing, the piston including at least one biasing device positioned to apply a constant force to the bellows to expel a medical fluid from the cartridge.

22. The portable infusion device of claim 21, the housing including a housing end configured to releasably secure the cartridge end to the housing.

23. The portable infusion device of claim 21, the cartridge end including and inlet and an outlet.

24. A portable infusion kit comprising:
a portable infusion device including
a cylindrical housing;
an inflatable cartridge filled with medical fluid, the inflatable cartridge including an inflatable portion connected to a cartridge end, the inflatable portion sized to fit within the housing, and the cartridge end configured to be releasably secured to the housing when the bellows is inserted into the housing;
a piston located within the housing, the piston including at least one biasing device positioned to apply a constant force to the inflatable portion of the cartridge to expel the medical fluid from the cartridge, and
a retracting tool including a threaded rod, the threaded rod including a handle on one end and a nut configured to be releasably secured to the housing.

25. The portable infusion kit of claim 24, further comprising an overwrap to encase the cartridge in an enclosure comprising a gas barrier, the atmosphere in the enclosure being a desired gas mixture.

26. The portable infusion kit of claim 25, the housing end configured to releasably secure the retracting tool to the housing.

27. The portable infusion kit of claim 24, the retracting tool including a contact surface configured to engage and place the piston in a retracted position prior to insertion of the filled cartridge into the housing.

28. The portable infusion kit of claim 24, the housing including a locking mechanism that maintains the piston in a retracted position.

29. A cartridge for a portable infusion kit, the portable infusion kit including a portable infusion device including a hollow housing, the cartridge comprising:
a bellows defining an internal cavity,
a medical fluid within the cavity, the bellows shaped to be retained within the housing when containing the medical fluid,
a cartridge end configured to be secured to the housing when the bellows is inserted into the housing;
an overwrap comprising a gas barrier;
and an atmosphere within the barrier being a desired gas mixture.
